(19) United States
(12) Patent Application Publication
(10) Pub. No.: US 2022/0361999 A1
(43) Pub. Date: Nov. 17, 2022

(54) DRUG INJECTION DEVICE

(71) Applicants: MYO SEONG, Anyang-si (KR); SAMSUNG LIFE PUBLIC WELFARE FOUNDATION, Seoul (KR)

(72) Inventors: Boo Rak LEE, Gimpo-si (KR); Ji Won SEONG, Incheon (KR); Duk Lyul NA, Seoul (KR); Seung Hoon LEE, Seoul (KR); Jeong Min LEE, Seoul (KR); Woo Ram JUNG, Seoul (KR); Kap Soo KIM, Seoul (KR); Nak Soon PARK, Wonju-si (KR)

(73) Assignees: MYO SEONG, Anyang-si (KR); SAMSUNG LIFE PUBLIC WELFARE FOUNDATION, Seoul (KR)

(21) Appl. No.: 17/773,134

(22) PCT Filed: Oct. 29, 2020
(86) PCT No.: PCT/KR2020/014916

§ 371 Date: Apr. 29, 2022

(30) Foreign Application Priority Data
Oct. 29, 2019 (KR) 10-2019-0135621

(51) Int. Cl. A61D 7/00; A61M 39/24; A61B 5/00; A61B 90/11

(52) U.S. Cl. CPC A61D 7/00; A61M 39/24; A61B 5/6864; A61B 5/6882; A61B 90/11

(57) ABSTRACT

Disclosed is a drug injection device that is implanted between the skull and the subcutaneous layer of an animal. The disclosed drug injection device includes: a main body that is positioned on the skull and implanted and fixed in the subcutaneous layer; a guide member that guides a trocar such that the drug is injected into brain parenchyma inside the skull of the animal; a cover member that is installed inside the main body and connected to the guide member in the center and has a hole for guiding drug injection trocar so as to be inserted; and a sealing member that is installed between the main body and the cover member and prevents reverse flow of drugs and the introduction of foreign substances.

### FIELD OF THE INVENTION

The present invention relates to a drug injection device, and more particularly, to a drug injection device that is implanted and fixed in the skull of a small and medium-sized animal to repeatedly inject a drug.

### BACKGROUND OF THE INVENTION

In recent years, diseases that lower quality of life, in particular, brain-related diseases that increase social and economic costs have become a lot of social issues. For example, in the case of brain diseases such as degenerative brain diseases, mental disorders, brain tumors and strokes, oral administration and intravenous administration methods have been widely used. However, in terms of effects and side effects, a method of directly administrating a drug to the brain is 100 times or more effective than existing different methods, and has few side effects due to systemic circulation of the drug, which is advantageous.

On the other hand, there is a difficulty in that the drug targeting the brain should pass through the blood-brain barrier (BBB). In the case of stem cells, similarly, the stem cells should be effectively delivered to the brain for their function. In a case where only the delivery efficiency of stem cells is considered, direct transplantation of the stem cells through brain surgery is the best method, but there is a risk in that a patient should undergo neurosurgery and there are practical limitations in a case where repeated administration is required. Animal experiments are essential to prove the effect of repeated drug administration on various brain diseases.

Vertebrate animals used or bred for the purpose of experiments have been used for these animal experiments. In particular, in the case of small rodents, for example, mice and rats, among experimental animals, since the skull is small and the subcutaneous layer is thin, in a case where a drug injection device is mounted, the device may be exposed to the outside of the brain. In this case, the drug injection device exposed to the outside has a high probability of being damaged or lost by movement of an animal or being infected by external contamination. In the related art, the drug injection device is weak to external shocks and cannot be easily fixed, so that the drug injection device cannot be stably maintained during the experiment, thereby resulting in a limit in repeating drug administration. In addition, there is a problem in that the experiment cost increases since animals are frequently infected with bacteria and die during the experiment.

### SUMMARY OF THE INVENTION

To solve the above problems, an object of the present invention is to provide a drug injection device capable of being implanted and fixed in the skull of a small and medium-sized animal to repeatedly inject a drug.

According to an aspect of the invention, there is provided a drug injection device that is implanted in a skull, including: a main body that is positioned on the skull, is implanted and fixed in a subcutaneous layer, and has an accommodating space therein, in which an upper part thereof is sealed and a drug injection hole into which a trocar is inserted is formed at a central portion thereof; a cover member that is positioned in the accommodating space of the main body, has a guide hole that guides injection of a drug at a central portion thereof, and is fixed to the skull; a guide member that is connected to the guide hole, is positioned under the cover member, and guides the trocar; and a sealing member that is provided between the main body and the cover member, and prevents a reverse flow of the drug and introduction of foreign substances.

The main body may include: a body part; and a mounting part that extends from the body part, and has at least one fixing hole for mounting and fixing to the skull.

The mounting portion may be provided so that a side portion extending from an upper portion of the main body to the skull is formed to be inclined.

The drug injection device may further include: at least one fixing member that is inserted into the fixing hole to fix the skull and the main body.

A coupling groove may be formed in the accommodating space of the main body, and a coupling protrusion may be formed on the cover member to be locked with the coupling groove.

The main body may satisfy at least one of the following Conditional Expressions 1 and 2.

0.4≤D₁/H₁23 10 <Conditional Expression 1>

3≤L₁≤15 [mm] <Conditional Expression 2>

Here, D₁ represents an upper outer diameter of the main body, H₁ represents a height of the main body, and L₁ represents a horizontal length of the main body.

The cover member may satisfy the following Conditional Expression 3.

0.6≤D₂/H₂≤100 <Conditional Expression 3>

Here, D₂ represents an outer diameter of the cover member, and H₂ represents a height of the cover member.

The guide member may have a curve at an end thereof and may satisfy the following Conditional Expression 4.

1≤L₁≤10 [mm] <Conditional Expression 4>

Here, L₁ represents a length of the guide member.

The sealing member may satisfy the following Conditional Expression 5.

0.1≤L₃≤3 [mm] <Conditional Expression 5>

Here, L₃ represents a height of the sealing member.

At least one of the main body, the cover member, the sealing member, and the guide member may be formed of polyetheretherketone, and the fixing member may be formed of a titanium material.

The drug injection device according to the present invention enables an animal to perform daily activities without an immune rejection reaction in a state of being implanted in the animal brain for a long period of time, and is implanted in the subcutaneous layer of the animal brain and thus does not protrude upward, thereby making it possible to prevent the drug injection device from falling off due to an external shock such as movement of the animal.

In addition, the drug injection device according to the present invention has an advantage that the cover member and the main body are primarily coupled to improve a fixing force even before the fixing member is fastened, thereby preventing separation even after long-term use.

Further, the drug injection device according to the present invention can guide the drug to be repeatedly injected by accurately positioning a drug target point using the guide member.

Furthermore, since the drug injection device according to the present invention includes the sealing member, it is possible to prevent penetration of contaminants into the brain due to widening of the drug injection hole through repeated operations.

### BRIEF DESCRIPTION OF THE DRAWINGS

FIG. 1 is a schematic view showing a state where a drug injection device according to an embodiment of the present invention is mounted on the skull of an experimental animal.

FIG. 2 is a view showing a state where the drug injection device of FIG. 1 is being implanted and a state where the implantation of the drug injection device is completed.

FIG. 3 is a perspective view showing a drug injection device according to an embodiment of the present invention.

FIG. 4 is a cross-sectional view of the drug injection device according to the embodiment of the present invention.

FIG. 5 is a schematic view for illustrating a coupling relationship between a main body of the drug injection device and a cover member thereof according to the embodiment of the present invention.

FIG. 6 is an exploded front view schematically showing a drug injection device according to another embodiment of the present invention.

FIG. 7 is a schematic view showing a modified example of a guide member of FIG. 4.

FIG. 8 is a partial cross-sectional perspective view showing an example in which a filter is added to a sealing member of the drug injection device according to the embodiments of the present invention.

FIG. 9A is a view showing a fixing member of the drug injection device according to the embodiments of the present invention.

FIG. 9B is a schematic perspective view showing a head part of the fixing member shown in FIG. 9A.

### DESCRIPTION OF EXEMPLARY EMBODIMENTS

Hereinafter, a drug injection device according to embodiments of the present invention will be described in detail with reference to the accompanying drawings. The accompanying drawings are shown by partially exaggerating or simplifying configurations of the invention for convenience of explanation and understanding of the invention.

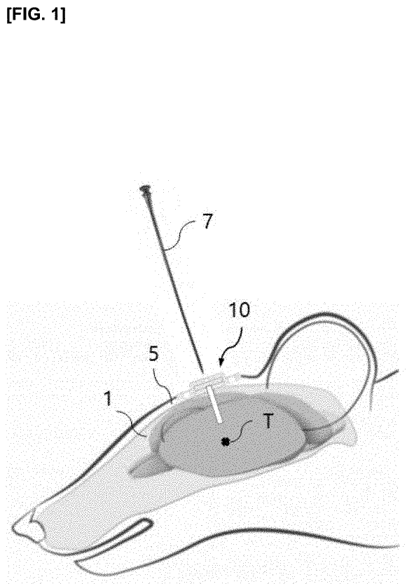

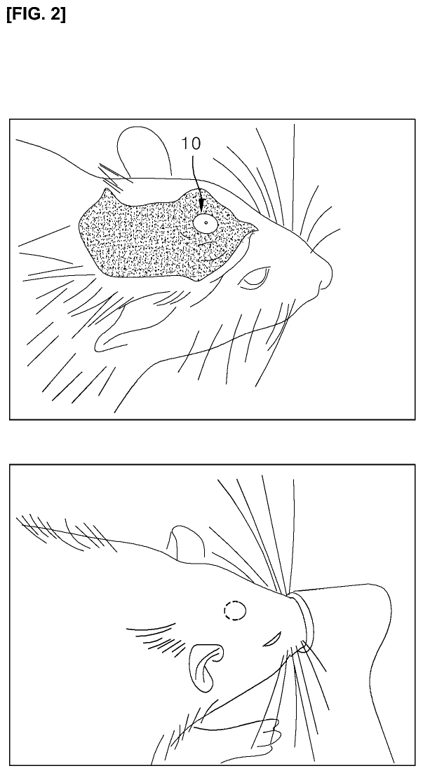

FIG. 1 is a schematic view showing a state where a drug injection device according to an embodiment of the present invention is mounted on the skull of an experimental animal, and FIG. 2 is a view showing a state where the drug injection device of FIG. 1 is being implanted and a state where the implantation of the drug injection device is completed.

Referring to FIG. 1, a drug injection device 10 according to an embodiment of the present invention is implanted and fixed in a subcutaneous layer 5 in a skull 1 of an experimental animal. The drug injection device 10 includes a trocar 7 provided at the center thereof for drug injection. Referring to FIG. 2, the upper view shows a state where the drug injection device 10 is being implanted in the skull of a mouse. The lower view shows a state where the scalp covers the drug injection device 10 after the drug injection device 10 is implanted. As understood from the drawing, the animal may perform daily activities without an immune rejection reaction in a state where the drug injection device 10 is implanted in the brain of the animal for a long period of time. In addition, since the drug injection device 10 is implanted into the subcutaneous layer of the brain of the animal and does not protrude upward, it is possible to prevent the drug injection device 10 from being separated and lost due to an external shock such as kicking of the animal.

Here, the trocar 7 may include an injection needle, an examination needle, or the like. In addition, various devices and equipment (for example, a stimulation lead, an ablation probe, a catheter, an injection or fluid delivery device, a biopsy needle, an extraction tool, or the like) other than the trocar 7 may be provided to perform a diagnostic and/or treatment process.

Drugs capable of being injected during an animal experiment using the drug injection device 10 according to the present invention may include all kinds of drugs necessary for various brain disease-related animal experiments. For example, dementia-related chemicals such as amyloid hypothesis (substance that inhibits β-amyloid protein production), AP protein aggregation inhibitor, tau aggregation inhibitor, cholinesterase inhibitor, NMDA receptor or antagonist, cholinergic precursor, antioxidant, or diabetes treatment drugs; stem cells such as human umbilical cord blood cells, human umbilical cord blood-derived mesenchymal stem cells, neural stem cells, or bone marrow stem cells; drugs for treating psychiatric diseases such as Parkinson's disease, depression, or schizophrenia; brain tumor and stroke-related therapeutic drugs, and the like, may be included.

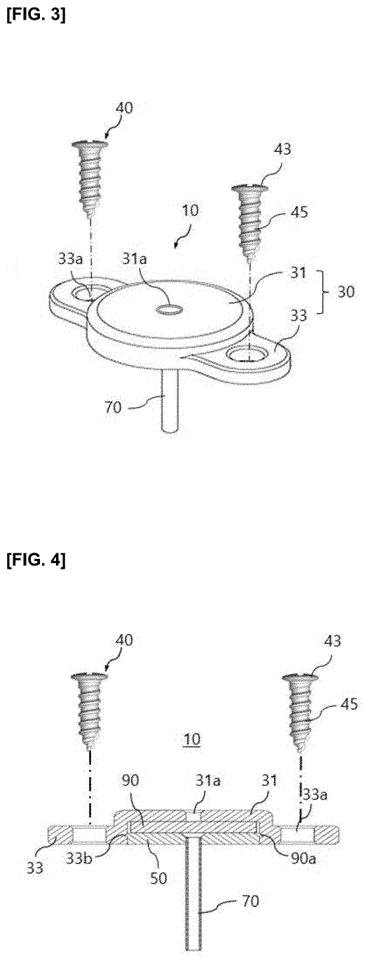

FIG. 3 is a perspective view showing the drug injection device according to the embodiment of the present invention, and FIG. 4 is a cross-sectional view showing the drug injection device according to the embodiment of the present invention.

Referring to FIGS. 3 and 4, the drug injection device 10 according to the embodiment of the present invention may include a main body 30, a cover member 50, a guide member 70, a sealing member 90, and a fixing member 40. The main body 30 has an accommodating space 31b therein, in which an upper part thereof is sealed and a drug injection hole 31a into which a trocar 7 is inserted is formed at a central portion thereof. The main body 30 may include a body part 31 and a mounting part 33, which may have a general hat shape. The mounting part 33 may be positioned at at least two points to be stably mounted on the skull 1. The fixing member 40 is inserted into a fixing hole 33a of the mounting part 30 to fix the main body 30 to the skull 1. The cover member 50 is positioned in the accommodating space of the main body 30, has a guide hole 50a that guides drug injection at a central portion thereof, and is fixed to the skull 1. The guide member 70 extends from the guide hole 50a, and is positioned under the cover member 50 to guide the trocar 7. The sealing member 90 is provided between the main body 30 and the cover member 50, and prevents a reverse flow of a drug and introduction of foreign substances.

The main body 30 may satisfy at least one of the following Expressions 1 and 2.

0.4≤D₁/H₁≤10 <Conditional Expression 1>

3≤L₁≤15 [mm] <Conditional Expression 2>

Here, D₁ represents an upper outer diameter of the main body, H₁ represents a height of the main body, and L₁ represents a horizontal length of the mounting part 33.

In Expression 1, in a case where D₁/H₁ is smaller than 0.4, an external size of the main body 30 excessively decreases, so that the durability and operability may be lowered. Contrarily, in a case where D₁/H₁ is greater than 10, an external size of the main body 30 excessively increases, so that the drug injection device 10 may protrude to the outside of the subcutaneous layer 5, thereby resulting in a risk of infection. Accordingly, under the condition of Expression 1, it is possible to implant the drug injection device 10 in the subcutaneous layer 5 without pressing the brain of a small rodent, and to confirm the position of the main body 30 by tactile sense in a case where the drug is repeatedly injected.

In Expression 2, in a case where L₁ is smaller than 3 mm, the length of the mounting part 33 is excessively short, so that it is difficult to fasten the fixing member 40. Further, in Expression 2, in a case where L₁ is greater than 15 mm, the length of the mounting part 33 is excessively long compared with the size of the brain of the small rodent, so that there is a concern that the drug injection device may compress the brain. Accordingly, under the condition of Expression 2, the drug injection device can have such an optimal size as to be fixed without being exposed to the outside and without compressing the brain of the small rodent after implantation in the subcutaneous layer 5.

FIG. 5 is a schematic view for illustrating a coupling relationship between a main body of the drug injection device and a cover member thereof according to the embodiment of the present invention.

Referring to FIG. 5, the body part 31 of the drug injection device 10 according to the embodiment of the present invention has an accommodating space 31b on a lower (inner) side thereof, and may accommodate at least one of the cover member 50, the guide member 70, and the sealing member 90. Further, in the body part 31, a drug injection hole 31a into which the trocar 7 is inserted may be formed at a central portion thereof. The drug injection hole 31a has a shape that becomes narrower downward, like a funnel, so that the drug can be easily introduced into the guide member 70. The mounting part 33 extends from the body part 31, and may have at least one fixing hole 33a for mounting and fixing to the skull 1. The cover member 50 is positioned under the main body 30, and may have a guide hole 50a that is connected to the guide member 70 at a central portion thereof and guides insertion of the trocar 7.

Referring to FIG. 5, in the accommodating space 31b of the main body 30 according to the present embodiment, a coupling groove 33b may be formed along an inner periphery thereof. Further, a coupling protrusion 50b may be formed along an outer periphery of the cover member 50. The coupling groove 33b and coupling protrusion 50b may be engaged with each other in the accommodating space 31b of the main body 30. This configuration shown in the drawing is not limiting, and different coupling configurations may be provided in the accommodating space 31b of the main body 30 and the cover member 50. Accordingly, since the main body 30 can be primarily coupled to the cover member 50 inserted into the skull 1, even in a case where the fixing member 40 is lost or even before the fixing member 40 is fastened, it is possible to stably combine the main body 30 and the cover member 50 to enhance a fixing force, thereby preventing separation thereof even after long-term use.

The cover member 50 may satisfy the following Expression 3.

0.6≤D₂/H₂≤100 <Conditional Expression 3>

Here, D₂ represents an outer diameter of the cover member, H₂ represents a height of the cover member.

In Expression 3, in a case where D₂/H₂ is smaller than 0.6, the space of the cover member 50 where the sealing member 90 is accommodated is excessively small, so that even if the size of the sealing member 90 is manufactured to be small, it is difficult to secure the function of the cover member 50. Further, in Expression 3, in a case where D₂/H₂ is greater than 100, the cover member 50 may push the main body 30 by the tension of the sealing member 90 to weaken the fixing force. Accordingly, under the condition of Expression 3, the cover member 50 can have such an optimal size as to be positioned on the skull 1 to prevent a reverse flow of the injected drug and to support the sealing member 90 that is positioned above the cover member 50.

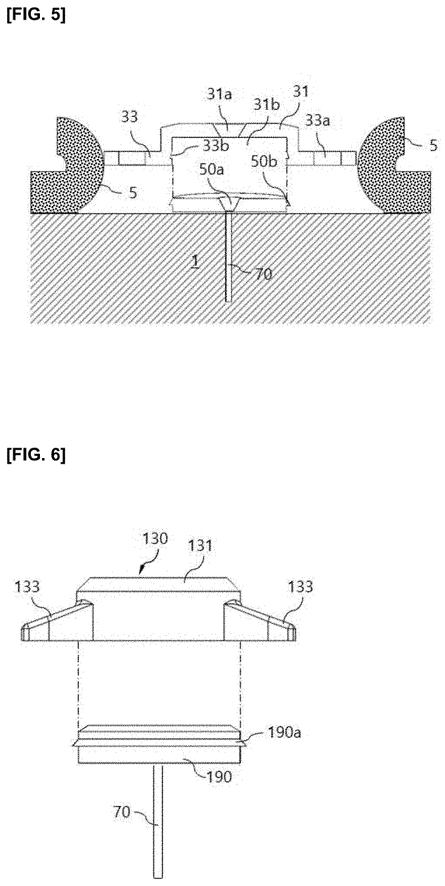

FIG. 6 is an exploded front view schematically showing a drug injection device according to another embodiment of the present invention.

Referring to FIG. 6, in the drug injection device according to the present embodiment, a mounting part 133 may have a side portion that extends from an upper portion to a lower portion of a main body 130 to form an inclination. Accordingly, it is possible to prevent the main body 30 from being bent by a force applied on both sides when the fixing member 40 is fastened to the mounting part 33. In addition, since the fixing member 40 is screw-coupled in an oblique direction to fit an inclined surface portion, it is possible to easily perform leveling with the inclined surface mounting part 33. The cover member 190 has a coupling protrusion 190a formed at a middle region of the side surface thereof, and can be prevented from being separated from the main body 130 after being accommodated in the body part 131. In the drug injection device according to the present embodiment, other components except for the main body 130 and the cover member 190 are the same as in that shown in FIG. 5, detailed description thereof will be not repeated.

The guide member 70 may guide the trocar 7 so that the drug is delivered to a target point T in brain parenchyma inside the skull 1 of the animal. Referring to FIG. 3, the trocar 7 may pass through the drug injection hole 31a to reach the guide member 70, and may deliver the drug to the final target point T. The guide member 70 can guide the trocar 7 to accurately position the drug target point T so that the drug can be repeatedly injected.

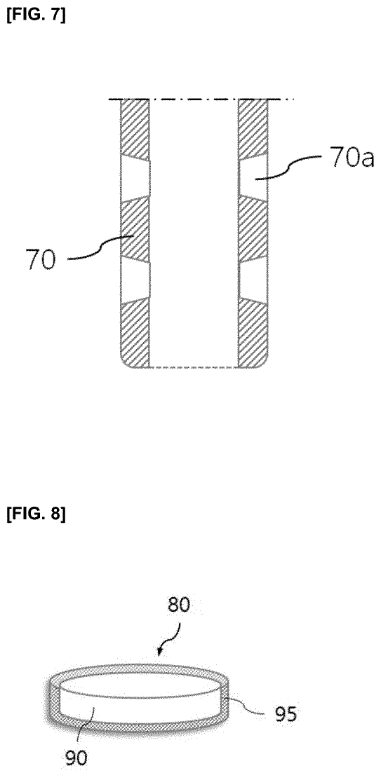

FIG. 7 is a schematic view showing a modified example of the guide member of FIG. 4.

The guide member 70 may have a curved end, and may satisfy the following Expression 4.

1≤L₂≤10 [mm] <Conditional Expression 4>

Here, L₂ represents a length of the guide member.

In Expression 4, in a case where L₂ is smaller than 1 mm, it is difficult to guide the trocar 7 to the target point in inserting the trocar 7 in order to inject the drug into the brain parenchyma. In Expression 4, in a case where L₂ is greater than 10 mm, the guide member may penetrate the skull beyond the target point. Accordingly, under the condition of Expression 4, the guide member 70 can have an optimal length in consideration of the insertion depth into the skull of small and medium-sized animals for experimentation.

Referring to FIG. 7, according to another embodiment of the present invention, the guide member 70 may have at least one spray groove 70a so that the drug is not accumulated at an end thereof. The spray groove 70a can spray the drug to be evenly distributed at the target point T without being accumulated at the end of the guide member 70.

The sealing member 90 is provided between the main body 30 and the cover member 50, and prevents a reverse flow of a drug and introduction of foreign substances. The sealing member 90 may satisfy the following Expression 5.

0.1≤L₃≤3 [mm] <Conditional Expression 5>

Here, L₃ represents a height of the sealing member.

In Expression 5, in a case where L₃ is smaller than 0.1 mm, the function of the sealing member 90 for completely blocking the brain parenchyma and the outside may be lost. Further, in Expression 5, in a case where L₃ is greater than 3 mm, the thickness of the sealing member 90 is excessively thick, so that the trocar 7 may be bent rather than being inserted. Under the condition of Expression 5, the sealing member 90 can be positioned between the main body 30 and the cover member 50, and can have an optimal size such that the sealing member 90 can be accommodated in the body part 31.

In the drug injection device 10, at least one of the main body 30, the cover member 50, the sealing member 90, and the guide member 70 may be formed of polyetheretherketone, and the fixing member 40 may be formed of a titanium material. However, the present invention is not limited thereto, and any material that is bio-implantable and MRI-applicable may be variously applied.

The sealing member 90 may contain a first mixture obtained by mixing 55 to 60% by weight of siloxane, silicone, dimethyl and vinyl groups, 25 to 30% by weight of silane amine, 1.1.1-trimethylaminetrimethylsilyl and silica hydrolysis products, and 5 to 7% by weight of siloxane silicone, dimethyl and methylvinyl; and a second mixture obtained by mixing 55 to 60% by weight of siloxane, silicone, dimethyl and vinyl groups, 25 to 30% by weight of silane amine, 1.1.1-trimethylaminetrimethylsilyl and silica hydrolysis products, 5 to 7% by weight of siloxane silicone, dimethyl and methylvinyl, and 1 to 5% by weight of silicon siloxane, dimethyl and methyl hydrogen.

The sealing member 90 may be characterized in that the first mixture and the second mixture satisfy the following Expression 6.

1≤M₂/M₁≤2 <Conditional Expression 6>

Here, M₁ represents the first mixture, and M₂ represents the second mixture.

In Expression 6, in a case where M₂ /M₁ is smaller than 1, the density of the mixture is excessively low, so that the mixture may be hardened over time or particles thereof may come off. Further, in Expression 6, in a case where M₂ /M₁ is greater than 2, the density of the mixture is excessively high, so that it may be difficult to insert the trocar. Accordingly, under the condition of Expression 6, the sealing member 90 can have an appropriate magnification of the first mixture and the second mixture, and can provide an optimal ratio such that the occurrence of bubbles on the surface is small and the particles of the mixture are not injected into the brain parenchyma when the trocar 7 is injected.

Referring to FIG. 8, the drug injection device 10 according to another embodiment of the present invention may further include a filter membrane 95 such that the sealing member 90 prevents introduction of foreign substances. The filter membrane 95 may have a mesh shape, and may be integrated with the sealing member 90 to form an integrated sealing filter member 80 to be easily mounted at once. With this configuration, the sealing filter member 80 can prevent introduction of foreign substances and bacteria from the outside, and also, can prevent a reverse flow of the drug.

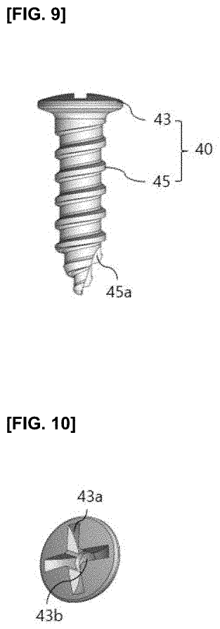

FIG. 9A is a view showing a fixing member of the drug injection device according to the embodiments of the present invention, and FIG. 9B is a schematic perspective view showing a head part of the fixing member shown in FIG. 9A.

Referring to FIGS. 9A and 9B, the fixing member 40 of the drug injection device 10 according to the embodiments of the present invention may be screw-fixed to the skull 1 through the fixing hole 33a of the mounting part 33. Here, at least one fixing member 40 may be used, and in a case where they are respectively fixedly mounted on both sides of the main body 33, the fixing force may increase. The fixing member 40 may include a body part 45 that is implanted and fixed in the skull and a head part 43 connected to the body part 45. The body part 45 and the head part 43 may satisfy at least one of the following Expressions 7 and 8.

1≤D₃/D₄<10 <Conditional Expression 7>

1≤L₅/L₄≤10 <Conditional Expression 8>

Here, D₃ represents an outer diameter of the head part, D₄ represents an outer diameter of the body part, L₄ represents a length of the head part, and L₅ represents a length of the body part.

In Expressions 7 and 8, in a case where D₃/D₄ and L₅/L₄ are smaller than 1, since the size of the body part 45 is larger than that of the head part 43, it is difficult to perform screwing, and in a case where D₃/D₄ and L₅/L₄ are greater than 10, it is difficult to secure a screw shape. Accordingly, under the conditions of Expressions 7 and 8, the fixing member 40 can have a screw shape that is most suitable for being placed in the skull 1 of the small animal to fix the mounting part 33.

The head part 43 may have a cross-shaped identification groove 43a at the center thereof so as to be horizontally engaged with a fastening tool (not shown). A concave engaging groove 43b may be formed at the center of the identification groove 43a to be engaged with the fastening tool at a correct position without magnetism. To this end, the fastening tool may be formed with a protrusion that matches the engaging groove 43b at a leading end thereof. That is, the identification groove 43a has a shape matching the cross-shaped protrusion of the fastening tool without magnetism, so that the fixing member 40 can be identified at once. That is, the engaging groove 43b is positioned at the center of the identification groove 43a and matches the protrusion of the fastening tool, thereby performing horizontal engagement without magnetism.

The identification groove 43a and the engaging groove 43b may satisfy the following Expression 9.

0.06≤D₅/L₆≤12 <Conditional Expression 9>

Here, L₆ represents a width of the identification groove, and D₆ represents a diameter of the engaging groove.

In Expression 9, in a case where D₅/L₆ is smaller than 0.06, the diameter of the engaging groove 43b is excessively small, it may be difficult to perform the engagement with the fastening tool, and in a case where D₅/L₆ is greater than 12, it may be difficult to perform the identifying function of the identification groove 43a. Accordingly, under the condition of Expression 9, the identification groove 43a and the engaging groove 43b can identify the fastening tool to be engaged with the fastening tool at a correct position without magnetism.

The body part 45 is implanted in the skull, and has a tapered thread to the end thereof, which may be formed with at least one pressure reducing groove 45a that satisfies the following Expression 10.

1<L₅/L₇≤50 <Conditional Expression 10>

Here, L₅ represents a length of the head part, and L₇ represents a length of a section in which the pressure reducing groove is formed.

In Expression 10, in a case where L₅/L₇ is smaller than 1, the fixing member 40 may be easily implanted but may be easily removed to weaken the fixing force, and in a case where L₅/L₇ is greater than 50, the function of the pressure reducing groove 45a may be weakened. Under the condition of Expression 10, the pressure reducing groove 45a can minimize damage to the skull due to pressure applied when the thread of the fixing member 40 is implanted in the skull.

A method of mounting the drug injection device 10 according to the present invention will be described in detail with reference to FIGS. 1 to 10.

A pre-operative preparation step is performed through MRI imaging using a navigation device (not shown) (S10). The pre-operative preparation step (S10) may include a step of inputting a location using a navigation probe and transmitting an image and a photo to an MRI machine (S11). The pre-operative preparation step may include a step of finding a location of a target point T in the brain of an animal on the basis of the MRI image or photo and marking a plurality of sections serving as a reference in the subcutaneous layer 5 of the animal (S13). The type of the navigation probe may be classified into a measurement probe and a verification probe. Using the verification probe, it is possible to mark the subcutaneous layer 5 of the animal after checking a path before cutting. The pre-operative preparation step may include a step of cutting the subcutaneous layer 5 of the animal located at the marking site in the shape of, for example, “L”, “¬” or “S” (S15). The pre-operative preparation step may include a step of spreading a cut portion to secure a space for a burr hole, and tying and pulling the cut subcutaneous layer 5 using a thread for operation to fix the cut subcutaneous layer 5 (S17). Here, it is possible to perform fixing and spreading using a pair of forceps or the like. Further, the pre-operative preparation step may include a step of securing a field of view by inhaling foreign substances, blood, or the like using a suction catheter together with physiological saline as necessary (S17a). In addition, the pre-operative preparation step may include a step of removing bone debris or residues with tweezers (forceps) after making the burr hole on the cut and exposed skull 1 (S19).

Then, a step of implanting the drug injection device 10 in the burr hole portion may be performed (S30). Specifically, the implanting step (S30) is a step of implanting the cover member 50 to which the guide member 70 is coupled to the burr hole portion, and may include a step of making the guide member 70 pass through the brain parenchyma of the animal so that the tip of the guide member 70 approaches the target point T (S31). Here, the guide member 70 should be inserted as carefully as possible so as not to damage the tissues of the brain parenchyma. After implanting the cover member 50 to the burr hole portion, the implanting step may include a step of inserting the sealing member 90 above the cover member 50 (S33). The implanting step may include a step of coupling or locking the coupling groove 33b formed in the accommodating space 31b of the main body 30 and the coupling protrusion 50b formed in the cover member 50 with the sealing member 90 interposed therebetween (S35). The implanting step may include a step of fixing the fixing member 40 to each of at least two fixing holes 33a formed in the main body 30 so that the drug injection device 10 is not separated from the skull 1 (S37).

Then, a step of injecting a drug to the target point T through the drug injection device 10 may be performed (S50). Specifically, the drug injection step (S50) may include a step of making the trocar 7 containing the drug sequentially pass through the drug injection hole 31a at the center of the main body 30, the sealing member 90, the guide hole 50a at the center of the cover member 50, and the guide member 70 to reach the final target point T to administer the drug (S51). Here, since the drug injection hole 31a and the guide hole 50a respectively have a shape that becomes narrower downward like a funnel, the drug can be easily introduced into the guide member 70. In addition, the drug injection step may include a step of making the trocar 7 containing the drug sequentially pass through the drug injection hole 31a at the center of the main body 30, the sealing member 90, the guide hole 50a at the center of the cover member 50, and the guide member 70 to reach the final target point T, thereby repeating administration multiple times over a long period of time (S53). Here, the drug injection step may include a step of evenly distributing the drug at the target point T using at least one spray groove 70a that prevents accumulation of the drug at the end of the guide member 70 (S55).

The above-described embodiments are merely exemplary, and various modifications and equivalents thereof may be made by those of ordinary skill in the art to which the present invention pertains. Accordingly, the true technical protection scope of the present invention should be determined by the technical idea of the invention disclosed in claims.

1. A drug injection device that is implanted in a skull, comprising:
a main body that is positioned on the skull, is implanted and fixed in a subcutaneous layer, and has an accommodating space on a lower side thereof, in which an upper part thereof is sealed and a drug injection hole into which a trocar is inserted is formed at a central portion thereof;
a cover member that is positioned to cover the accommodating space of the main body, has a guide hole that guides injection of a drug at a central portion thereof, and is fixed to the skull;
a guide member that is connected to the guide hole, is positioned under the cover member, and guides the trocar; and
a sealing member that is provided between the main body and the cover member, and prevents a reverse flow of the drug and introduction of foreign substances,
wherein the guide member is formed with at least one spray groove for preventing the drug from accumulating at an end thereof so that the drug is evenly distributed to a target point.

2. The drug injection device according to claim 1,
wherein the main body includes:
a body part; and
a mounting part that extends from the body part, and has at least one fixing hole for mounting and fixing to the skull.

3. The drug injection device according to claim 2,
wherein the mounting part is provided so that a side portion extending from an upper portion of the main body to the skull is formed to be inclined.

4. The drug injection device according to claim 2, further comprising:
at least one fixing member that is inserted into the fixing hole to fix the skull and the main body, and is formed of a titanium material.

5. The drug injection device according to claim 1,
wherein a coupling groove is formed in the accommodating space of the main body, and a coupling protrusion is formed on the cover member to be locked with the coupling groove.

6. The drug injection device according to claim 1,
wherein the main body satisfies at least one of the following Conditional Expressions 1 and 2:
0.4≤D1/H1≤10 <Conditional Expression 1>
3≤L1≤15 [mm] <Conditional Expression 2>
where D1 represents an upper outer diameter of the main body, H1 represents a height of the main body, and L1 represents a horizontal length of the main body.

7. The drug injection device according to claim 1,
wherein the cover member satisfies the following Conditional Expression 3:
0.6≤D2/H2≤100 <Conditional Expression 3>
where D2 represents an outer diameter of the cover member, and H2 represents a height of the cover member.

8. The drug injection device according to claim 1,
wherein the guide member has a curve at an end thereof and satisfies the following Conditional Expression 4:
1≤L1≤10 [mm] <Conditional Expression 4>
where L1 represents a length of the guide member.

9. The drug injection device according to claim 1,
wherein the sealing member satisfies the following Conditional Expression 5:
031≤L3≤3 [mm] <Conditional Expression 5>
where L3 represents a height of the sealing member.

10. The drug injection device according to claim 1,
wherein at least one of the main body, the cover member, the sealing member, and the guide member is formed of polyetheretherketone.

11. The drug injection device according to claim 2,
wherein the main body satisfies at least one of the following Conditional Expressions 1 and 2:
0.4≤D1/H1≤10 <Conditional Expression 1>
3≤L1≤15 [mm] <Conditional Expression 2>
where D1 represents an upper outer diameter of the main body, H1 represents a height of the main body, and L1 represents a horizontal length of the main body.
